(19) United States
(12) Patent Application Publication
(10) Pub. No.: US 2007/0014831 A1
(43) Pub. Date: Jan. 18, 2007

(54) Biodegradable occlusive device with moisture memory

(76) Inventors: Hsing-Wen Sung, HsinChu (TW); Mei-Chin Chen, Taipei County (TW); Hosheng Tu, Newport Beach, CA (US)

Correspondence Address: HOSHENG TU, 15 RIEZ, NEWPORT BEACH, CA, 92657-0116, US

(21) Appl. No.: 11/180,498

(22) Filed: Jul. 12, 2005

(51) Int. Cl. A61K 47/48; C08G 63/91; C08G 63/48; A61F 2/02

(52) U.S. Cl. 424/426; 525/54.1; 525/54.2

(57) ABSTRACT

The present invention relates to a biodegradable occlusive device and methods for treating aneurysm of a patient comprising deploying a flexible biodegradable occlusive device with a moisture memory and a controlled biodegradation, and deploying a retaining stent for preventing the occlusive device from being inadvertently dislodged from the sac.

### FIELD OF THE INVENTION

The present invention generally relates to a biodegradable biological material or device crosslinked with a crosslinking agent characterized by moisture memory. More particularly, the present invention relates to a crosslinked flexible biodegradable material as an embolization device for treating an aneurysm sac in a patient, followed by controlled bioresorption of the device in situ.

### BACKGROUND OF THE INVENTION

Crosslinking of biological molecules is often desired for optimal effectiveness and biodurability in biomedical applications. For example, collagen, which constitutes the structural framework of biological tissue, has been extensively used for manufacturing bioprostheses and other implanted structures, such as vascular grafts, wherein it provides a good medium for cell infiltration and proliferation. However, biomaterials derived from collagenous tissue must be chemically modified and subsequently sterilized before they can be implanted in humans. The fixation, or crosslinking, of collagenous tissue increases strength and reduces antigenicity and immunogenicity. In some aspects, the medical devices made of solidifiable biological material are feasible as a biodegradable occlusive device with inherent moisture memory.

Clinically, the biological material has to be fixed with a crosslinking agent or chemically modified and subsequently sterilized before they can be implanted in humans. Some purposes of fixing biological material are to reduce antigenicity and/or immunogenicity and mitigate enzymatic degradation. Various crosslinking agents have been used in fixing biological material. These crosslinking agents are mostly synthetic chemicals such as formaldehyde, glutaraldehyde, dialdehyde starch, glyceraldehydes, cyanamide, diimides, diisocyanates, dimethyl adipimidate, carbodiimide, and epoxy compound. However, these chemicals are all highly cytotoxic which may impair the biocompatibility of biological tissue. Of these, glutaraldehyde is known to have allergenic properties, causing occupational dermatitis and is cytotoxic at concentrations greater than 10-25 ppm and as low as 3 ppm in tissue culture. It is, therefore, desirable to provide a crosslinking agent suitable for use in biomedical applications that is within acceptable cytotoxicity and that forms stable and biocompatible crosslinked products.

An example of a genipin-crosslinked heart valve is reported by Sung et al., a co-inventor of the present invention, (Journal of Thoracic and Cardiovascular Surgery vol. 122, pp. 1208-1218, 2001) entitled Reconstruction of the right ventricular outflow tract with a bovine jugular vein graft fixed with a naturally occurring crosslinking agent (genipin) in a canine model, the entire contents of which are incorporated herein by reference. Sung et al. herein discloses genipin and its crosslinking ability to a collagen-containing biological tissue heart valve used successfully in an animal implantation study.

U.S. Pat. No. 5,085,629 issued on Feb. 4, 1992, the entire contents of which are incorporated herein by reference, discloses a biodegradable, biocompatible, resorbable infusion stent comprising a terpolymer of L(−)lactide, glycolide, and epsilon-caprolactone. This invention includes a method for treating ureter obstructions or impairments by utilizing a biodegradable, biocompatible, resorbable infusion stent, and a method for controlling the speed of resorption of the stent. A ureter stent that is made of a biodegradable and biocompatible material would assure its safe and innocuous disappearance without the need for a second surgical procedure for its removal after it has completed its function.

Embolization is generally understood as a therapeutic introduction of a substance into a vessel in order to occlude it. It is a treatment used in cases such as patent ductus arteriosus, major aortopulmonary collateral arteries, pulmonary arteriovenous malformations, venovenous collaterals following venous re-routing operations, occlusion of Blalock-Taussig shunts, and occlusion of coronary arteriovenous fistulas. The use of embolization therapy in the intracranial region of the brain for embolizing aneurysms or fistulas is generally accepted.

U.S. Pat. No. 6,790,218, entire contents of which are incorporated herein by reference, discloses a device for occluding an anatomical defect comprising a wire member formed of a non-biodegradable shape memory alloy, the member having a first predetermined unexpanded shape and a second predetermined expanded shape, wherein the unexpanded shape is substantially linear and the expanded shape is substantially conical.

U.S. Pat. No. 6,569,190, entire contents of which are incorporated herein by reference, discloses a method for treating an aneurysm in a mammalian patient comprising identifying the vascular site of an aneurysmal sac and inhibiting systemic blood flow into the aneurysmal sac by filling at least a portion of the sac with a non-particulate agent and a fluid composition which solidifies in situ.

Aneurysms results from a vascular disease wherein the arterial wall weakens and, under pressure due to blood flow, the arterial wall balloons. Eventual rupture of the ballooned arterial wall is associated with high morbidity and mortality rates. Intracranial aneurysms are of particular concern because surgical procedures to treat these aneurysms before rupture are often not feasible and further because rupture of these aneurysms can have devastating results on the patient even if the patient survives rupture. Accordingly, treatment protocols for intracranial aneurysms may be prophylactic in nature to inhibit rupture of the aneurysm rather than to inhibit bleeding from the ruptured aneurysm.

U.S. Pat. No. 6,723,112, entire contents of which are incorporated herein by reference, discloses an implantable medical device for at least partially obstructing a neck portion of a vascular aneurysm, comprising an occlusion subassembly comprising a base section and at least one lateral protrusion fixedly attached to the base section, and a therapeutic agent disposed upon at least one portion of the occlusion subassembly, the therapeutic agent being a bioactive material, the bioactive material being a biologically absorbable suture material that encourages cell growth.

U.S. Pat. No. 6,595,876, entire contents of which are incorporated herein by reference, discloses an expandable endovascular prosthesis comprising a first expandable portion being made from a plastically deformable material and expandable with a radially outward force to cause plastic deformation, and a second expandable portion attached to the first expandable portion, the second expandable portion being made from a plastically deformable material and is larger after expansion.

U.S. Pat. No. 5,776,097, entire contents of which are incorporated herein by reference, discloses a device for treating an intracranial vascular aneurysm located on an intracranial blood vessel, the device comprising a catheter with an inflation balloon, means for visualizing the blood vessel lumen adjacent the aneurysm lumen, and means for delivering a liquid sealing agent to the lumen of the aneurysm.

In accordance with the present invention there is provided crosslinked collagen-containing or chitosan-containing biological devices which have shown to exhibit moisture memory and controlled, predetermined biodegradation for optimal embolization function.

### SUMMARY OF THE INVENTION

In general, it is an object of the present invention to provide a biological substance configured and adapted for drug slow release and biodegradation. In one aspect of the present invention, the biological substance may be a cardiovascular stent or implant. The “biological substance” is herein intended to mean a substance made of drug-containing biological material that is, in one preferred embodiment, solidifiable upon change of environmental condition(s) and is biocompatible post-crosslinking with a crosslinker, such as genipin, its derivatives, analog (for example, aglycon geniposidic acid), stereoisomers and mixtures thereof. In one embodiment, the crosslinker may further comprise epoxy compounds, dialdehyde starch, glutaraldehyde, formaldehyde, dimethyl suberimidate, carbodiimides, succinimidyls, diisocyanates, acyl azide, reuterin, ultraviolet irradiation, dehydrothermal treatment, tris(hydroxymethyl)phosphine, ascorbate-copper, glucose-lysine and photo-oxidizers, and the like. The “biological material” is intended herein to mean collagen, gelatin, elastin, chitosan, NOCC (N, O, carboxylmethyl chitosan), fibrin glue, biological sealant, and the like that could be crosslinked with a crosslinker (also known as a crosslinking agent).

Some aspects of the invention relate to a flexible bioresorbable biological material comprising a moisture memory and a controlled bioresorption, wherein the material is crosslinked with a crosslinking agent having a degree of crosslink that is correlated to a controllable bioresorption rate configured to enable the controlled bioresorption when implanted in a patient. The controlled bioresorption is correlated to predetermined or pre-configured biodegradation phenomenon.

In one embodiment, the flexible bioresorbable biological material with the moisture memory is in a first shape at a wet moisture state, re-configurable to a second shape at a dry state, and reversible to the first shape after contacting moisture.

In one embodiment, the flexible bioresorbable biological material is selected from the group consisting of collagen, gelatin, elastin, chitosan, NOCC, chitosan-alginate complex, and combinations thereof. In a further embodiment, the flexible bioresorbable biological material is crosslinked with a crosslinking agent selected from the group consisting of genipin, its analog, derivatives, and combinations thereof, aglycon geniposidic acid, epoxy compounds, and combinations thereof. In a further embodiment, the flexible bioresorbable biological material is crosslinked with a crosslinking agent selected from the group consisting of dialdehyde starch, glutaraldehyde, formaldehyde, dimethyl suberimidate, carbodiimides, succinimidyls, diisocyanates, reuterin, acyl azide, and combinations thereof.

In one embodiment, the flexible bioresorbable biological material is crosslinked with a crosslinking agent, the crosslinking agent comprising at least one ether group or ethyl compound. In another embodiment, the flexible bioresorbable biological material is crosslinked with a crosslinking agent, the crosslinking agent comprising ethylene glycol diglycidyl ether. In a further embodiment, the flexible bioresorbable biological material further comprises at least one bioactive agent.

Some aspects of the invention relate to a flexible elongate biodegradable device for treating an aneurysm of a patient, the device being characterized with a moisture memory and a controlled biodegradation, wherein the device comprises a first configuration in a wet state sized and configured to snugly fill an aneurysm sac of the aneurysm; the device having a second configuration in a dry state configured to be loaded in a delivery apparatus; and the device reversing to the first configuration after being deployed from the delivery apparatus into the sac.

In one embodiment, the flexible elongate biodegradable device for treating an aneurysm of a patient is made of a polymer material containing at least one amino group, wherein the material is crosslinked with a crosslinking agent having a degree of crosslink that is correlated to a controllable biodegradation rate configured to enable the controlled biodegradation in a patient.

In one embodiment, the flexible elongate biodegradable device for treating an aneurysm of a patient is made of a biological material selected from the group consisting of collagen, gelatin, elastin, chitosan, NOCC, chitosan-alginate complex, and combinations thereof. In another embodiment, the device may comprise at least one bioactive agent or at least one blood occluding agent.

In one embodiment, the flexible elongate biodegradable device for treating an aneurysm of a patient is crosslinked with a crosslinking agent having a degree of crosslink, wherein the degree of crosslink is correlated to a controllable biodegradation rate configured to enable the controlled biodegradation, wherein the crosslinking agent is selected from the group consisting of genipin, its analog, derivatives, and combination thereof, aglycon geniposidic acid, epoxy compounds, dialdehyde starch, glutaraldehyde, formaldehyde, dimethyl suberimidate, carbodiimides, succinimidyls, diisocyanates, reuterin, acyl azide, ethylene glycol diglycidyl ether, and combinations thereof.

Some aspects of the invention relate to a method of treating an aneurysm sac of a patient, comprising steps of: providing a flexible elongate biodegradable device with a moisture memory and a controlled biodegradation, wherein the device comprises a first configuration in a wet state sized and configured to snugly fill the aneurysm sac; delivering the device to about the aneurysm sac, wherein the device comprises a second configuration in a dry state configured to be loadable in a delivery catheter during the delivery step; deploying the device at the aneurysm sac, wherein the device reversely transforms to the first configuration after being deployed from the catheter and contacting moisture; and the device starting a process of biodegradation in situ following the controlled biodegradation of the present invention. In one embodiment, the method further comprises a step of placing a retaining (supporting) stent at a neck of the aneurysm sac configured for preventing the occlusive device from being inadvertently dislodged from the sac, wherein the stent is biodegradable.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 is chemical structures of glutaraldehyde and genipin that are used in the chemical treatment examples of the current disclosure.

FIG. 2 is a proposed crosslinking mechanism for a crosslinker, genipin (GP) with collagen intermolecularly and/or intramolecularly.

FIG. 3 is a biodegradable stent with mesh type tubular configuration.

FIG. 4 is one embodiment of a spiral (coil) biodegradable stent according to the principles of the invention.

FIG. 5 is another embodiment of an open-ring biodegradable stent according to the principles of the invention.

FIG. 6 is an occlusive device with moisture memory at a configuration of the wet state.

FIG. 7 is a perspective view of an occlusive device during a later stage of the delivery phase.

FIG. 8 is a perspective view of an occlusive device at a conclusive stage of the delivery phase.

FIG. 9 is a perspective view of an occlusive device and retaining implant for treating an aneurysm sac of a patient.

FIG. 10 shows some general trends of controlled bioresorption rates with respect to a parameter of degrees of crosslink.

### DETAILED DESCRIPTION OF EXEMPLARY EMBODIMENTS

The following detailed description is of the best presently contemplated modes of carrying out the invention. This description is not to be taken in a limiting sense, but is made merely for the purposes of illustrating general principles of embodiments of the invention.

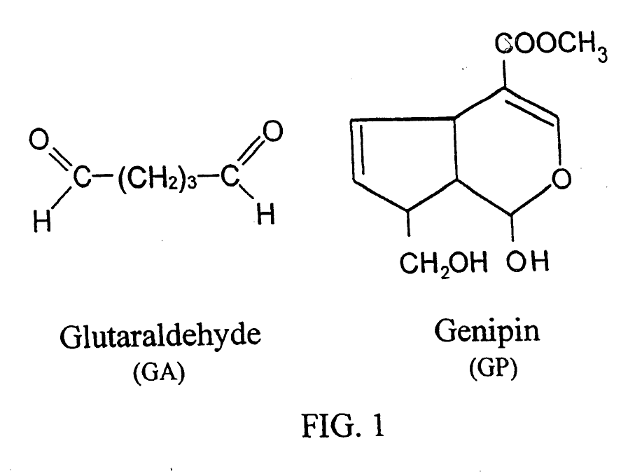

“Genipin” in this invention is meant to refer to the naturally occurring compound as shown in FIG. 1 and its derivatives, analog, stereoisomers and mixtures thereof.

“Crosslinking agent” is meant herein to indicate a chemical agent that could crosslink (form a bridge between) two molecules, such as formaldehyde, glutaraldehyde, dialdehyde starch, glyceraldehydes, cyanamide, diimides, diisocyanates, dimethyl adipimidate, carbodiimide, genipin, proanthocyanidin, reuterin, and epoxy compound.

“Biological material” is herein meant to refer to collagen (collagen extract, soluble collagen, collagen solution, or other type of collagen), elastin, gelatin, fibrin glue, biological sealant, chitosan (including N, O, carboxylmethyl chitosan), chitosan-containing and other collagen-containing biological material. For an alternate aspect of the present invention, the biological material is also meant to include a solidifiable biological substrate comprising at least a crosslinkable functional group, such as an amino group or the like.

A “biological implant” refers to a medical device which is inserted into, or grafted onto, bodily tissue to remain for a period of time, such as an extended-release drug delivery device, drug-eluting stent, vascular or skin graft, orthopedic prosthesis, such as bone, ligament, tendon, cartilage, and muscle.

In particular, the crosslinked collagen-drug or chitosan-drug device or compound with drug slow release capability may be suitable in treating atherosclerosis or for other therapeutic applications. In one aspect of the invention, it is provided a biodegradable medical device comprising at least one bioactive agent and at least one biological material. The biodegradable medical device is thereafter crosslinked with a crosslinking agent.

“Drug” in this invention is meant to broadly refer to a chemical molecule(s), biological molecule(s) or bioactive agent providing a therapeutic, diagnostic, or prophylactic effect in vivo. “Drug” and “bioactive agent” (interchangeable in meaning) may comprise, but not limited to, synthetic chemicals, biotechnology-derived molecules, herbs, cells, genes, growth factors, health food and/or alternate medicines. In the present invention, the terms “drug” and “bioactive agent” are used interchangeably.

The “biological substance” is herein intended to mean a substance made of drug-containing biological material that is, in one preferred embodiment, solidifiable upon change of environmental condition(s) and is biocompatible after being crosslinked with a crosslinker, such as genipin, epoxy compounds, dialdehyde starch, glutaraldehyde, formaldehyde, dimethyl adipimidate, carbodiimide, proanthocyanidin, or the like. Some aspects of the invention provide a crosslinked biodegradable stent or implant comprising at least one bioactive agent and being crosslinked with certain means for crosslinking the biological material.

In a co-pending patent application Ser. No. 10/827,673 filed on Apr. 19, 2004, entitled “Crosslinkable Biological Material and Medical Use”, it is disclosed crosslinkable biological material with controlled biodegradation properties. In a co-pending patent application Ser. No. 10/916,170 filed Aug. 11, 2004, entitled “Drug-Eluting Biodegradable Stent”, it is disclosed a biodegradable implant having drug-loading capability. The entire contents of these two co-pending applications are incorporated herein by reference.

Preparation and Properties of Genipin

Genipin, shown in FIG. 1, is an iridoid glycoside present in fruits (Gardenia jasmindides Ellis). It may be obtained from the parent compound geniposide which may be isolated from natural sources as described elsewhere (Sung H W et al., in Biomaterials and Drug Delivery toward New Millennium, Eds K D Park et al., Han Rin Won Publishing Co., pp. 623-632, (2000)). Genipin, the aglycone of geniposide, may be prepared from the latter by oxidation followed by reduction and hydrolysis or by enzymatic hydrolysis. Alternatively, racemic genipin may be prepared synthetically.

Genipin has a low acute toxicity, with LD₅₀ i.v. 382 mg/kg in mice. It is therefore much less toxic than glutaraldehyde and many other commonly used synthetic crosslinking agents. As described below, genipin is shown to be an effective crosslinking agent for treatment of biological materials intended for in vivo biomedical applications, such as prostheses and other implants, wound dressings, and substitutes.

It is one object of the present invention to provide a drug-collagen-genipin and/or drug-chitosan-genipin compound that is loaded onto a cardiovascular stent enabling drug slow-release to the surrounding tissue, or to the lumen of the bodily cavity. In one preferred embodiment, the compound is loaded onto the outer periphery of the stent enabling drug slow-release to the surrounding tissue. In another embodiment, the compound is fabricated as a stent enabling drug slow-release to the surrounding tissue.

The genipin derivatives and/or genipin analog may have the following chemical formulas (Formula 1 to Formula 4):

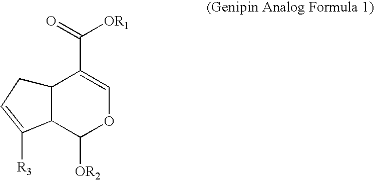

in which
R₁ represents lower alkyl;
R₂ represents lower alkyl, pyridylcarbonyl, benzyl or benzoyl;
R₃ represents formyl, hydroxymethyl, azidomethyl, 1-hydroxyethyl, acetyl, methyl, hydroxy, pyridylcarbonyl, cyclopropyl, aminomethyl substituted or unsubstituted by (1,3-benzodioxolan-5-yl)carbonyl or 3,4,5-trimethoxybenzoyl, 1,3-benzodioxolan-5-yl, ureidomethyl substituted or unsubstituted by 3,4,5-trimethoxyphenyl or 2-chloro-6-methyl-3-pyridyl, thiomethyl substituted or unsubstituted by acetyl or 2-acetylamino2-ethoxycarbonyethyl, oxymethyl substituted or unsubstituted by benzoyl, pyridylcarbonyl or 3,4,5-trimethoxybenzoyl;
provided that R₃ is not methyl formyl, hydroxymethyl, acetyl, methylaminomethyl, acetylthiomethyl, benzoyloxymethyl or pyridylcarbonyloxymethyl when R₁ is methyl, and

its pharmaceutically acceptable salts, or stereoisomers.

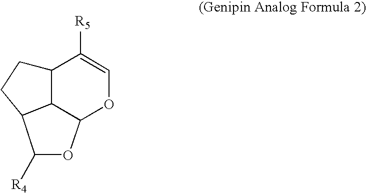

in which
R₄ represents lower alkoxy, benzyloxy, benzoyloxy, phenylthio, C₁˜C₁₂ alkanyloxy substituted or unsubstituted by t-butyl, phenyl, phenoxy, pyridyl or thienyl;
R₅ represents methoxycarbonyl, formyl, hydroxyiminomethyl, methoxyimino-methyl, hydroxymethyl, phenylthiomethyl or acetylthiomethyl;
provided that R₅ is not methoxycarbonyl when R₁₄ is acetyloxy; and
its pharmaceutically acceptable salts, or stereoisomers. 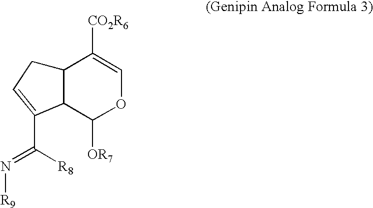
R₆ represents hydrogen atom, lower alkyl or alkalimetal;
R₇ represents lower alkyl or benzyl;
R₈ represents hydrogen atom or lower alkyl;
R₉ represents hydroxy, lower alkoxy, benzyloxy, nicotinoyloxy, isonicotinoyloxy, 2-pyridylmethoxy or hydroxycarbonylmethoxy;
provided that R₉ is not hydroxy or methoxy when R₆ is methyl and R₈ is hydrogen atom; and
its pharmaceutically acceptable salts, or stereoisomers.

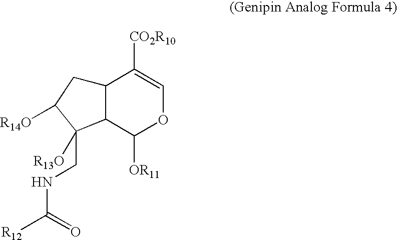

in which
R₁₀ represents lower alkyl;
R₁₁ represents lower alkyl or benzyl;
R₁₂ represents lower alkyl, pyridyl substituted or unsubstituted by halogen, pyridylamino substituted or unsubstituted by lower alkyl or halogen, 1,3-benzodioxolanyl;
R₁₃ and R₁₄ each independently represent a hydrogen atom or join together to form isopropylidene; and
its pharmaceutically acceptable salts, or stereoisomers.

In a co-pending patent application Ser. No. 10/924,538, filed Aug. 24, 2004, entitled “Medical use of reuterin”, it is disclosed that reuterin (B-hydroxypropionaldehyde) as a naturally occurring crosslinking agent can react with the free amino groups of biological material of the present invention. Further, in a co-pending patent application Ser. No. 10/929,047, filed Aug. 27, 2004, entitled “Medical use of aglycon geniposidic acid”, it is disclosed that aglycon geniposidic acid as a naturally occurring crosslinking agent can react with the free amino groups of biological material of the present invention.

In one embodiment of the present invention, it is disclosed that a method for treating tissue of a patient comprising, in combination, providing a drug-containing biological material to be shaped as a medical device (for example, an embolization occlusive device), chemically treating the drug-containing biological material with a crosslinking agent, and delivering the medical device to a target tissue for releasing the drug and treating the tissue by occluding an aneurysmal sac. The compound (such as collagen-drug-genipin compound, the chitosan-drug-genipin compound, or combinations thereof) and methods of manufacture as disclosed and supported by the following examples produce new and unexpected results and hence are unobvious from the prior art. The medical device can be a stent, a non-stent implant or prosthesis for the intended drug slow release. In a preferred aspect, the stent application with the compound (such as collagen-drug-genipin compound, the chitosan-drug-genipin compound, or combinations thereof) comprises medical use in lymphatic vessel, gastrointestinal tract (including the various ducts such as hepatic duct, bile duct, pancreatic duct, etc.), urinary tract (ureter, urethra, etc.), and reproductive tract (i.e., uterine tube, etc.). In one aspect, the non-stent implant may comprise annuloplasty rings, heart valve prostheses, venous valve bioprostheses, occlusive devices, orthopedic implants, dental implants, ophthalmology implants, cardiovascular implants, and cerebral implants. In another aspect of the present invention, the target tissue may comprise vulnerable plaque, atherosclerotic plaque, tumor or cancer, brain tissue, vascular aneurysm, vascular vessel or tissue, vascular vessel defect, orthopedic tissue, ophthalmology tissue or the like. The vulnerable plaque is the atherosclerotic plaque that is vulnerably prone to rupture in a patient.

In another embodiment of the present invention, it is disclosed a biological substance for treating tissue of a patient with drug slow release, wherein the biological substance is made of drug-containing biological material that may be solidifiable upon change of environmental condition(s) and is biocompatible after being crosslinked with a crosslinker, such as genipin, epoxy compounds, dialdehyde starch, dimethyl adipimidate, carbodiimide, glutaraldehyde, or the like. In one embodiment, the drug for embolization purposes can be a clotting agent, such as protamine, fibrin, collagen, or the like. In an alternate embodiment, the biological substance is without added drugs, wherein the biological material possesses some therapeutic functions, such as anti-inflammatory, anti-infection, and the like.

In still another embodiment of the present invention, it is disclosed that a method for treating tissue of a patient comprising, in combination, mixing a drug with a biological material, pre-forming the drug containing biological material as a medical device, chemically treating the pre-formed biological material with a crosslinking agent, and delivering the crosslinked biological material to a lesion site for treating the tissue. In one alternate embodiment, the method further comprises a step of solidifying the drug-containing biological material, either before the delivering step (ex vivo) or after the delivering step (in situ).

It is some aspect of the present invention that the method may further comprise chemically linking the drug with the biological material through a crosslinker, wherein the drug comprises at least a crosslinkable functional group, for example, an amino group.

In the present invention, the terms “crosslinking”, “fixation”, “chemical modification”, and “chemical treatment” for tissue or tissue material are used interchangeably.

FIG. 1 shows chemical structures of glutaraldehyde and genipin that are used in the chemical treatment examples of the current disclosure. Other crosslink agents may equally be applicable for collagen-drug-genipin and/or chitosan-drug-genipin compound disclosed herein.

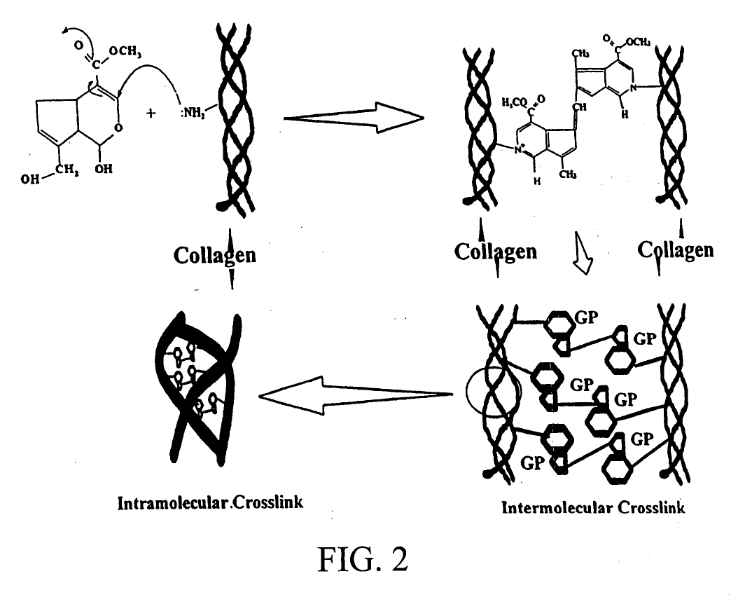

Other than genipin and glutaraldehyde, the crosslinking agent that may be used in chemical treatment of the present invention may include formaldehyde, dialdehyde starch, glyceraldehydes, cyanamide, diimides, diisocyanates, dimethyl adipimidate, carbodiimide, and epoxy compound. FIG. 2 shows a proposed crosslinking mechanism for a crosslinker, genipin (GP) with collagen intermolecularly and/or intramolecularly.

Glutaraldehyde has been used extensively as a crosslinking agent for fixing biologic tissues. By means of its aldehyde functional groups, glutaraldehyde reacts primarily with the εC-amino groups of lysyl or hydroxylysyl residues within biologic tissues. The mechanism of fixation of biologic tissues or biologic matrix with glutaraldehyde can be found elsewhere. Polymerization of glutaraldehyde molecules in aqueous solution with observable reductions in free aldehyde have been reported previously (Nimni M E et al. in Nimni M E, editor. COLLAGEN. Vol. III. Boca Raton (Fla.); CRC Press 1998. pp. 1-38).

It is postulated that a substance (for example, a drug) having an amine or amino functional group may react with glutaraldehyde as illustrated above. By combining collagen, glutaraldehyde and a drug having an amine or amino group, the crosslinked compound may link collagen to the drug via glutaraldehyde as a crosslinker. Delivery means for delivering a device into a blood vessel of a patient is well known to one ordinary skilled in the art, for example, U.S. Pat. No. 5,522,836 issued on Jun. 4, 1996 and U.S. Pat. No. 5,980,514 issued on Nov. 9, 1999.

Several biocompatible plastic polymers or synthetic polymers have one or more amine group in their chemical structures, for example poly(amides) or poly(ester amides). The amine group may become reactive toward a crosslinker, such as glutaraldehyde, genipin or epoxy compounds. Therefore, it is contemplated that by combining a polymer having an amine group, glutaraldehyde and a drug having at least an amine or amino group, the crosslinked compound may have the polymer linked to the drug via glutaraldehyde as a crosslinker. Other crosslinkers are also applicable.

Genipin Crosslinking

It was reported by Sung H W (Biomaterials 1999;20:1759-72) that genipin can react with the free amino groups of lysine, hydroxylysine, or arginine residues within biologic tissues. A prior study reports that the structures of the intermediates, leading to a blue pigment produced from genipin and methylamine, the simplest primary amine. The mechanism was suggested that the genipin-methylamine monomer is formed through a nucleophilic attack by methylamine on the olefinic carbon at C-3 of genipin, followed by opening of the dihydropyran ring and attack by the secondary amino group on the resulting aldehyde group. The blue-pigment was thought formed through oxygen radical-induced polymerization and dehydrogenation of several intermediary pigments.

As disclosed by Sung H W (J Thorac Cardiovasc Surg 2001;122:1208-1218), the simplest component in the blue pigment was a 1:1 adduct. It was suggested that genipin reacts spontaneously with an amino acid to form a nitrogen iridoid, which undergoes dehydration to form an aromatic monomer. Dimerization occurs at the second stage, perhaps by means of radical reaction. The results suggest that genipin may form intramolecular and intermolecular crosslinks with cyclic structure within collagen fibers in biologic tissue (FIG. 2) or solidifiable collagen-containing biological material.

It is disclosed herein that genipin is capable of reacting with a drug having an amine or amino group. By combining collagen (or a biological material or matrix), genipin and the drug having an amine or amino group, the crosslinked compound may have collagen linked to the drug via genipin as a bridge crosslinker.

As disclosed and outlined in the U.S. Pat. No. 6,545,042, issued on Apr. 8, 2003, entitled “Acellular biological material chemically treated with genipin” by one of the present inventors, the degrees in inflammatory reaction in the animal studies for the genipin-fixed cellular and acellular tissue were significantly less than their glutaraldehyde-fixed counterparts. Additionally, it was noted that the inflammatory reactions for the glutaraldehyde-fixed cellular and acellular tissue lasted significantly longer than their genipin-fixed counterparts. These findings indicate that the biocompatibility of the genipin-fixed cellular and acellular tissue is superior to the glutaraldehyde-fixed cellular and acellular tissue. It is hypothesized that the lower inflammatory reactions observed for the genipin-fixed cellular and acellular tissue may be due to the lower cytotoxicity of their remaining residues, as compared to the glutaraldehyde-fixed counterparts. In a previous study, it was found that genipin is significantly less cytotoxic than glutaraldehyde (J Biomater Sci Polymer Edn 1999;10:63-78). The cytotoxicity observed for the glutaraldehyde-fixed cellular and acellular tissue seems to result from a slow leaching out of unreacted glutaraldehyde as well as the reversibility of glutaraldehyde-crosslinking. It was observed that when concentrations above 0.05% glutaraldehyde were used to crosslink materials, a persistent foreign-body reaction occurred (J Biomater Sci Polymer Edn 1999;10:63-78).

Some aspects of the invention relate to genipin-crosslinked gelatin as a drug carrier. Some aspects of the invention relate to genipin-crosslinked fibrin glue and/or biological sealant as a drug carrier. In one embodiment, it is provided a method for treating tissue of a patient comprising, in combination, loading a solidifiable drug-containing gelatin (or fibrin glue/biological sealant) onto an apparatus or medical device, solidifying the drug-containing gelatin, chemically treating the gelatin with a crosslinking agent, and delivering the medical device to the tissue for treating the tissue. Gelatin microspheres haven been widely evaluated as a drug carrier. However, gelatin dissolves rather rapidly in aqueous environments, making the use of gelatin difficult for the production of long-term drug delivery systems. Hsing and associates reported that the degradation rate of the genipin-crosslinked microspheres is significantly increased (J Biomed Mater Res 2003;65A:271-282). U.S. Pat. No. 6,045,570, the entire contents of which are incorporated herein by reference, discloses a non-fibrin biological sealant comprising gelatin slurry which includes milled gelatin powder, wherein the slurry may include Gelfoam™ powder mixed with a diluent selected from the group consisting of saline and water. In a further disclosure, the biological sealant may thrombin, or calcium.

U.S. Pat. No. 6,624,138, entitled “Drug-loaded Biological Material Chemically Treated with Genipin”, the entire contents of which are incorporated herein by reference, disclose a method for treating tissue of a patient comprising, in combination, mixing a drug with a solidifiable biological material, chemically treating the drug with the biological material with a crosslinking agent, loading the solidifiable drug-containing biological material onto a medical device, solidifying the drug-containing biological material; and delivering the medical device to a target tissue for treating the tissue.

### EXAMPLE #1 CHITOSAN

Dissolve chitosan powder in acetic acid at about pH 4. Chitosan (MW: about 70,000) was purchased from Fluka Chemical Co. of Switzerland. The deacetylation degree of the chitosan used was approximately 85%. Subsequently, adjust the chitosan solution to approximately pH 5.5 (right before it becomes gelled) with NaOH. Add in drug(s) of interest into the chitosan solution. While loading the drug-containing chitosan onto a stent, adjust the environment to pH 7 with NaOH to solidify the chitosan onto the stent. In another embodiment, the drug-containing chitosan is configured to become a stent or a coil-shaped implant by exposing to an environment of pH 7 to solidify the chitosan stent. In one embodiment, the process can be accomplished via a continuous assembly line step by providing gradually increasing pH zones as the device passes by. It is further treated with a crosslinking agent, for example genipin or epoxy compounds, to enhance the biodurability and biocompatibility. Note that the chemical formula for chitosan can be found in Mi F L, Tan Y C, Liang H F, and Sung H W, “In vivo biocompatibility and degradability of a novel injectable-chitosan based implant.” Biomaterials 2002;23:181-191, and is shown below.

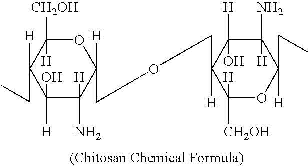

Chitosan is a copolymer of glucosamine and N-acetylglucosamine, derived from the natural polymer chitin, which is commercially available. Chitosan has been reported to be a potentially useful pharmaceutical material because of its good biocompatibility and low toxicity. Some aspects of the invention relate to a biodegradable stent or occlusive device made of a biological material selected from a group consisting of chitosan, collagen, elastin, gelatin, fibrin glue, biological sealant, and combination thereof. In a further embodiment, the stent is crosslinked with a crosslinking agent or with ultraviolet irradiation. In another embodiment, the stent is loaded with at least one bioactive agent.

### EXAMPLE #2 Chitosan stent

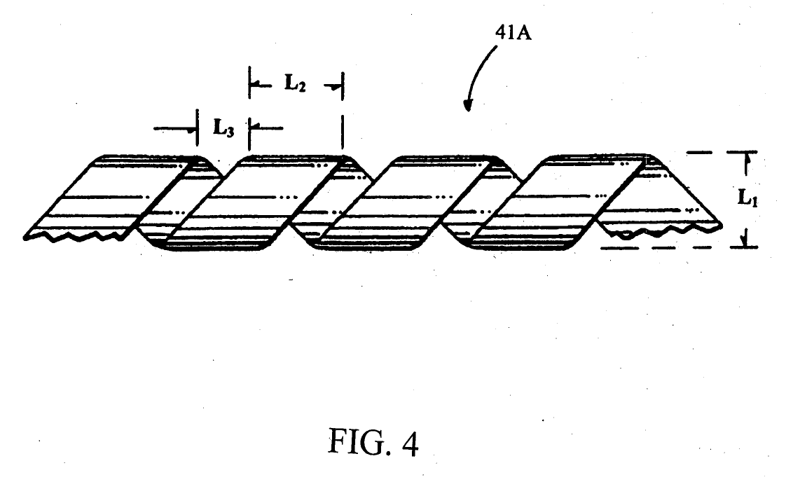

Dissolve chitosan powder in acetic acid at about pH 4 by dispersing 3 grams powder in 50 ml of water containing 0.5 wt % acetic acid. Chitosan (MW: about 70,000) was purchased from Fluka Chemical Co. (Buchs, Switzerland). The chitosan polymer solution was prepared by mechanical stirring at about 600 rpm for about 3 hours until all powder is dissolved. Subsequently, adjust the chitosan solution to approximately pH 5.5 (right before it becomes gelled) with NaOH. Add in at least one bioactive agent of interest into the chitosan solution. While loading the bioactive agent-containing chitosan onto a mold, adjust the environment to pH 7 with NaOH to solidify the chitosan to make a stent. In one example, the mold is a helically bendable hollow mold (such as the one made of silicone or polyurethane-silicone copolymer). During the solidification stage, the mold is promptly bent helically or spirally. After the chitosan is fully solidified, remove the mold to obtain a shaped chitosan pre-product. In another example, a cylindrical mold is used to make a cast chitosan film onto the inner surface of the cylindrical mold. During the solidification stage, the mold may be rotated at a desired speed, say, several hundred to several thousand rpm. The cylindrical film, after solidified, is thereafter cut by a spiral knife to make a spiral chitosan pre-product (as shown in FIG. 4). In a third example, the solidifiable solution is made into films, whereas the films are cut into strips of about 1-2 mm wide. These strips are then wound onto a mandrill and the helical pre-product are fabricated, wherein the fabrication method may comprise heat set or other change in the environment conditions (such as pH, temperature, or crosslinking).

In any case, the chitosan pre-product may be further treated with a crosslinking agent, for example genipin, to enhance the biodurability, biocompatibility, but retain certain desired biodegradability. In a preferred embodiment, the chitosan cylindrical film can be cut to make a double spiral or double helix pre-product. In another preferred embodiment, after the step of adjusting the chitosan solution to approximately pH 5.5 with NaOH, another substrate or biological material, such as collagen, gelatin, fibrin glue, biological sealant, elastin, NOCC (N, O, carboxylmethyl chitosan), chitosan-alginate complex, combination thereof, and the like, or phosphorylcholine may be promptly added and well mixed during the manufacturing process. U.S. Pat. 5,607,445, the entire contents of which are incorporated herein by reference, discloses a stent having helical and double helical configurations. In one embodiment, the resistance to enzymatic degradation of the biological elastin component in a biodegradable stent can be enhanced by treatment with a crosslinking agent, such as tannic acid (Isenburg J C et al., Biomaterials 2003).

Mi F L, Sung H W and Shyu S S in “Drug release from chitosan-alginate complex beads reinforced by a naturally occurring cross-linking agent” (Carbohydrate Polymers 2002;48:61-72), the entire contents of which are incorporated herein by reference, discloses drug controlled release characteristics of a chitosan-alginate complex as biological material which is crosslinkable with a crosslinking means for crosslinking the biological material and capable of being loaded with at least one drug or bioactive agent.

Fibrin glue is a two-component system of separate solutions of fibrinogen and thrombin/calcium. When the two solutions are combined, the resultant mixture mimics the final stages of the clotting cascade to form a fibrin clot. The fibrinogen component can be prepared extemporaneously from autologous, single-donor, or pooled blood. Of course, autologous blood carries essentially no risk of serologically transmitted disease but is also not practical for emergency situations. Fibrin glue is available in Europe under the brand names Beriplast™, Tisseel™, and Tissucol™. Fibrin glue has been used in a wide variety of surgical procedures to repair, seal, and attach tissues in a variety of anatomic sites. The advantage of fibrin glue over other adhesives, such as the cyanoacrylates, is that it is a natural biomaterial that is completely reabsorbed in 2 weeks to 4 weeks. However, the rate of resorption or biodegradation can be slowed down (for example, to about 2 months) via appropriate crosslinking enabling its use in the drug-eluting stents or embolization occlusive devices.

FIG. 4 shows one embodiment of a spiral (helical or coil) biodegradable stent 41A according to the principles of the invention (in one embodiment, as a retaining stent in an embolization system of the present invention). In one embodiment, the spiral stent 41A comprises a spiral film having a cylindrical diameter L₁, a film thickness, a film width L₂ and the spacing L₃ between two helical portions of the film. The film thickness is usually in the range of about 20 microns to 800 microns, preferably 100 to 500 microns. The film width L₂ is usually in the range of about 0.2 mm to 5 mm, preferably 0.5 to 2 mm. The spacing L₃ is usually in the range of about 0.5 to 5 mm, preferably between 0.5 and 2 mm. For non-coronary applications, the upper limit of the aforementioned dimensions could be several times higher. The cylindrical diameter L₁, of the spiral film may expand from a first diameter to a second diameter after the film absorbs liquid or water due to its swelling effect of the biological material used in making the biodegradable stent of the invention. On the other hand, the non-metallic stent made of synthetic polymer, such as non-biodegradable polymer or biodegradable polymer (for example, poly(L-lactic acid), polyglycolic acid, poly (D,L-lactide-co-glycolide), poly (ester amides), polycaprolactone, co-polymers thereof, and the like), the diameter change after absorbing liquid (such as water, plasma, or serum) is insignificant. The increase of the cylindrical diameter enhances the retention of the stent against the vessel wall. Some aspects of the invention relate to a biodegradable stent that has a first diameter before contacting water and a second diameter after contacting water, wherein the second diameter is at least 5% more than the first diameter. In one aspect of the invention, it is provided a biodegradable stent that has a first circumference length before contacting water and a second circumference length after contacting water, wherein the second circumference length is at least 5% more than the first circumference length.

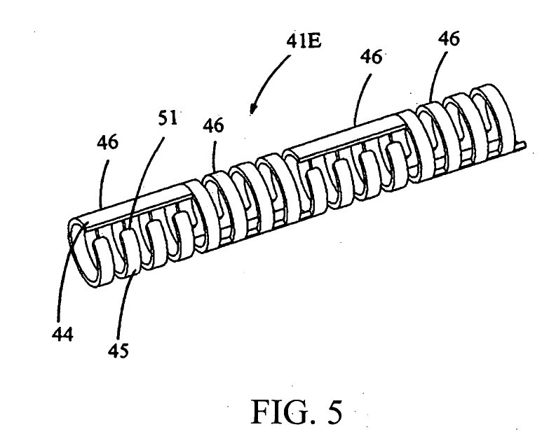

FIG. 5 shows another embodiments of an open-ring biodegradable stent 41E comprising a plurality of open-ring stent members 46 wherein the bases 44 of the stent members are secured to each other and oriented in a way that the open-ring end 51 of the first stent member 46 may point to a different direction from that of the second stent member. This is to facilitate a more balanced open ring arrangement of an open-ring biodegradable stent (in one embodiment, as a retaining stent in an embolization system of the present invention).

### EXAMPLE #3 EPOXY COMPOUNDS CROSSLINKING

Following the steps for making solidifiable chitosan solution or other biological solution optionally loaded with a bioactive agent in the previous example, the solution is cast to make a chitosan film pre-product. The film is thereafter cut by a knife to have a strip wrapped on a mandrill in a helical fashion to make a spiral pre-product or a double spiral pre-product. The pre-product is crosslinked with a polyepoxy compound, such as ethylene glycol diglycidyl ether, or a polyepoxy compound containing at least one ether group (such as —O—) shown below.

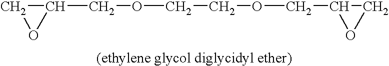

The ethylene glycol diglycidyl ether contains two ether groups, wherein the ether group has two branching compounds attached to the —O— that functions as a pivotal center for the branching compounds to relatively free swing about the —O—, enabling the crosslinked device with moisture memory. The device crosslinked with ethylene glycol diglycidyl ether crosslinker exhibits a first shape at a wet state, re-configurable to a second shape at a dry state, and reversible to the first shape after contacting moisture. Further, the device crosslinked with ethylene glycol diglycidyl ether crosslinker exhibits a degree of crosslink that is correlated to a controllable bioresorption rate configured to enable the controlled bioresorption.

With proper packaging and sterilization, the biodegradable stent is fabricated. Some aspects of the invention relate to a flexible bioresorbable biological material comprising a moisture memory and a controlled bioresorption, wherein the material is crosslinked with a crosslinking agent having a degree of crosslink that is correlated to a controllable bioresorption rate configured to enable the controlled bioresorption when implanted in a patient. In one embodiment, the material with the moisture memory is in a first shape at a wet state, re-configurable to a second shape at a dry state, and reversible to the first shape after contacting moisture. In another embodiment, the biological material is selected from a group consisting of collagen, gelatin, elastin, chitosan, NOCC, chitosan-alginate complex, and combinations thereof. The fixation details could be found elsewhere by Sung et al. (Sung H W, Chang Y, Liang I L, Chang W H and Chen Y C. “Fixation of biological tissues with a naturally occurring crosslinking agent: fixation rate and effects pfpH, temperature, and initial fixative concentration.” J Biomed Mater Res 2000;52:77-87).

### Example #4

Add drug in a NOCC solution at room temperature. The NOCC (named after “Nitrogen Oxygen carboxylmethyl chitosan”) is a chitosan derived compound that is pH sensitive and can be used in drug delivery. This NOCC is water soluble at pH 7. Pre-shape and crosslink the NOCC and drug by a crosslinking agent, for example genipin. This is a step of solidification. In one aspect of the present invention, after crosslinking, the shape of the drug containing NOCC can be made harder or permanent. The finished device slowly releases drug when in the body of a patient at a body pH.

In a separate study, we evaluated genipin-crosslinked chitosan membranes that were fabricated by means of a casting/solvent evaporation technique (Mi F L et al., J Biomater Sci Polymer Edn 2001;12(8):835-850). The crosslinked chitosan film which could be used to make a biodegradable spiral (coil) stent showed ultimate tensile strength values at about 50-55 MPa. The tensile strength of a dog-bone sample is considered indirectly correlated to the collapse pressure of a cylindrical type stent (Venkatraman S et al., Biomaterials 2003;24:2105-2111). The ultimate tensile strength of the crosslinked chitosan membranes is about equivalent to that of Venkatraman PLLA4.3 specimen of about 55 MPa (FIG. 4 in Biomaterials 2003;24:2105-21 11) The strain-at-fracture values for the crosslinked chitosan membranes range from about 9 to 22%, which overlaps the strain-at-fracture ranges of 8-12% for Venkatraman PLLA4.3 and PLLA8.4 specimens as shown in FIG. 5 in Biomaterials 2003;24:2105-2111. Further, the swelling ratio for the crosslinked chitosan membranes indicates its desired hydrophilicity as an implant.

### EXAMPLE #5

Taxol (paclitaxel) is practically water insoluble as some other drugs of interest in this disclosure. Therefore, first mechanically disperse paclitaxel in a collagen solution at about 4° C. Load the drug containing collagen onto a stent and subsequently raise the temperature to about 37° C. to solidify collagen fibers on the stent. The loading may comprise spray coating, dip coating, plasma coating, painting or other known techniques. The loading step may repeat a plurality of times. Subsequently, crosslink the coated stent with aqueous genipin or polyepoxy compound. The crosslinking on the drug carrier, collagen or chitosan, substantially modify the drug diffusion or eluting rate depending on the degree of crosslink as correlated to the biodegradation of the crosslinked drug carrier (collagen or chitosan).

### EXAMPLE #6

Sirolimus is used as a bioactive agent in this example. First, mechanically disperse sirolimus in a collagen solution at about 420 C. Load the sirolimus containing collagen onto a stent and subsequently raise the temperature to about 37° C. to solidify collagen fibers on the stent. The loading may comprise spray coating, dip coating, plasma coating, painting or other known techniques. The loading step may repeat a plurality of times, wherein each loading step is followed by a crosslinking step with predetermined crosslinking degree.

The resulting sirolimus containing stent with chemically crosslinked collagen is sterilized and packaged for clinical use. By way of example, one preferred sterilization condition may comprise 0.2% peracetic acid and 4% ethanol at room temperature for a period of 1 minute to a few hours. In one embodiment, the medical device of the invention is sterilized with a condition comprising a sterilant of peracetic acid about 0.1 to 5% and alcohol (preferably ethanol) about 1 to 20% at a temperature of 5 to 50° C. for a time of about 1 minute to 5 hours. In another embodiment, the device of the present invention can be sterilized before use by lyophilization, ethylene oxide sterilization, or sterilized in a series of ethanol solutions, with a gradual increase in concentration from 20% to 75% over a period of several hours. Finally, the drug-loaded devices are rinsed in sterilized saline solution and packaged. The drug carrier, collagen and chitosan, may be fully or partially crosslinked. In one aspect of the present invention, a partially crosslinked collagen/chitosan is biodegradable or bioerodible for embolization purposes.

As used herein, the term “biodegradable” refers to a material that is bioresorbable, whereas the material degrades and/or breaks down by enzymatic degradation upon interaction with a physiological environment into components that are metabolizable or excretable, over a period of time. In one aspect, the biodegradable polymer comprises a biodegradable linkage selected from the group consisting of ether groups, ester groups, carbonate groups, amide groups, anhydride groups, and orthoester groups. By way of example, poly(ester amides), particularly poly[(8-L-Leu-6)₃-(8-L-Lys(Bz))₁,], is well known to one skilled in the art which has been disclosed in U.S. Pat. No. 5,485,496 and elsewhere.

Embolization Device

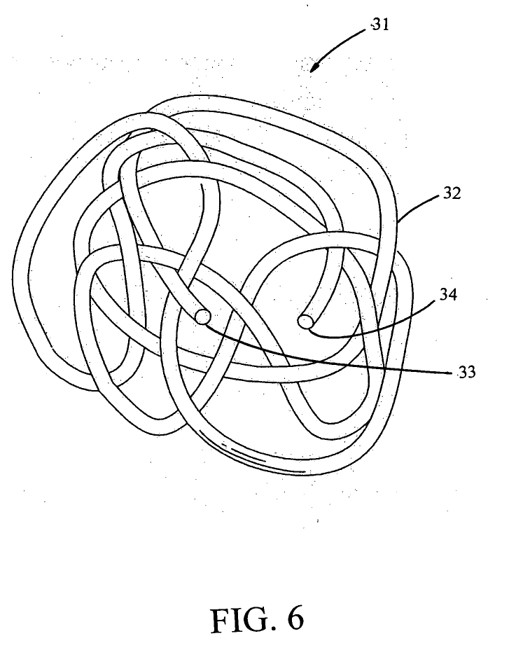

FIG. 6 shows a flexible embolization device 31 which may be delivered by a catheter to a pre-selected position within a blood vessel to thereby embolize a blood vessel or a blood vessel defect, such as an aneurysm or fistula. Some aspects of the invention relate to a vascular embolization system for treating a defect in a blood vessel comprising: a catheter having a proximal section, a distal section and an outer wall defining a lumen therethrough; a push rod type plunger slidably disposed within the lumen of the catheter having a proximal end and a distal end; and, an embolization device comprising an elongated random coil-type configuration with moisture memory; the embolization device being disposed within the lumen at the distal section of the catheter, the distal end of the push rod engages the embolization device such that distal movement of the push rod causes the embolization device to exit the lumen of the catheter at a pre-selected position (such as an aneurysmal sac) within the blood vessel.

“Moisture memory” is herein intended to define a material or device comprising a first configuration in a wet moisture state under neither external restriction nor compression, the device comprising a second configuration in a dry state under any predetermined confinement, such as configured essentially straight to be loadable in a delivery catheter, and the device reversing to the first configuration after contacting moisture or exposed to a wet state under neither external restriction nor compression. In one embodiment, the device at the wet state is sized and configured to be a configuration (31 as shown in FIG. 6) that could snugly fill an aneurysm sac of the aneurysm, wherein the device 32 has a distal end 33 and a proximal end 34.

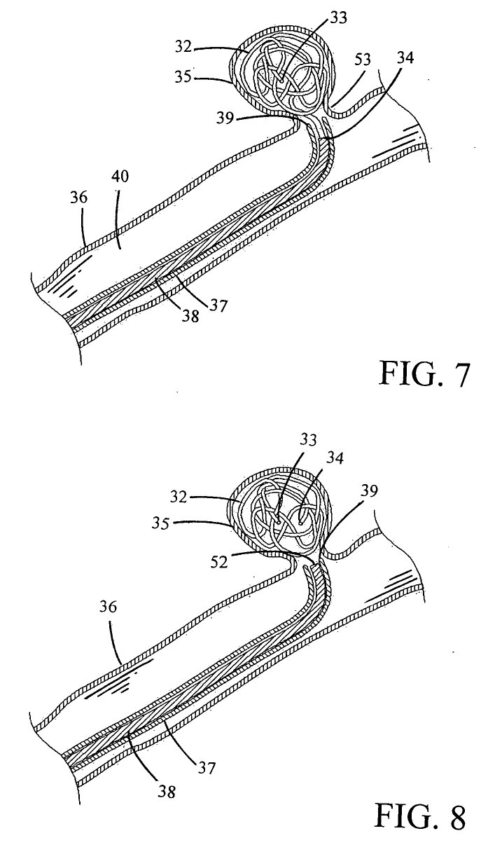

FIG. 7 shows a perspective view of an occlusive device during a later stage of the delivery phase, whereas FIG. 8 shows a perspective view of the occlusive device at a conclusive stage of the delivery phase. The occlusive device 32 in a second configuration (essentially straight) is loaded inside a delivery catheter 37 at about the distal tip 39 during a dry state in vivo. The catheter and the indwelled device is inserted through a vessel opening and is advanced along the blood vessel 36 to about the lesion site. The delivery catheter is sized and configured to move inside the lumen 40 of the blood vessel 36 atraumatically. Once approaching the neck 53 of the aneurysmal sac 35, the distal tip 39 is deflected to unload the distal end 33 of the device into the sac by advancing a plunger 38 inside the lumen of the catheter 37. The distal end 52 of the plunger continues to push the essentially straight device into the sac, wherein the device reverses to its first configuration as a result of moisture memory under wet conditions. Upon completing the pushing step, the proximal end 34 of the device detaches from the plunger 38 and forms a curled shape to snugly fill the sac.

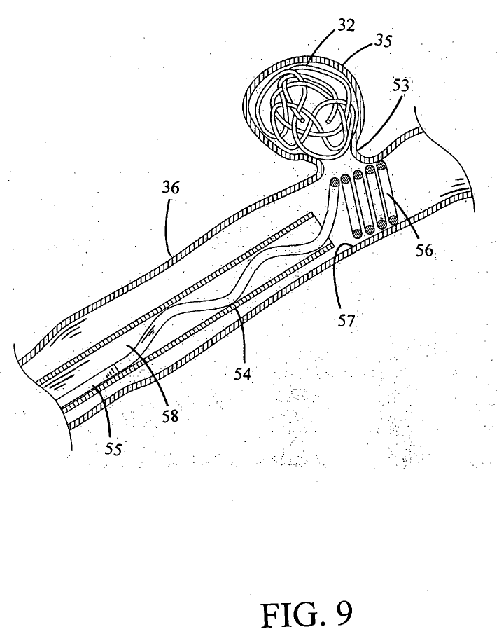

FIG. 9 shows a perspective view of a system including an occlusive device 32 inside a sac and a retaining implant 56 for treating an aneurysm sac 35 of a patient. The retaining implant or retaining stent 56 may comprise a coil type configuration to snugly fit the internal wall 57 of a vessel 36 about outside of the sac neck 53 so as to retain the embolization device in place. In one embodiment, the retaining stent 56 is usually loaded inside the lumen 58 of a stent delivery apparatus 54 for deployment. The delivery and deployment of a self-expanding vascular stent is well known to one ordinary skilled in the art.

In one embodiment, the embolization device is comprised of a radiopaque material for procedural viewing. In another embodiment, the embolization device is comprised of at least a therapeutic agent. After implanting an occlusive device at an aneurysmal sac, the embolization process starts to firm up the sac and mitigate further rupture possibility. Once the sac is embolized, the embolization device is no longer needed since it is a foreign material. The device would biodegrade gradually or become bioresorbable and leave no trace of device behind. In one embodiment, the biodegradation is a controlled bioresorption, wherein the material that has a degree of crosslink being correlated to a controllable bioresorption rate is configured to enable the controlled bioresorption in a patient.

Biodegradable Stent

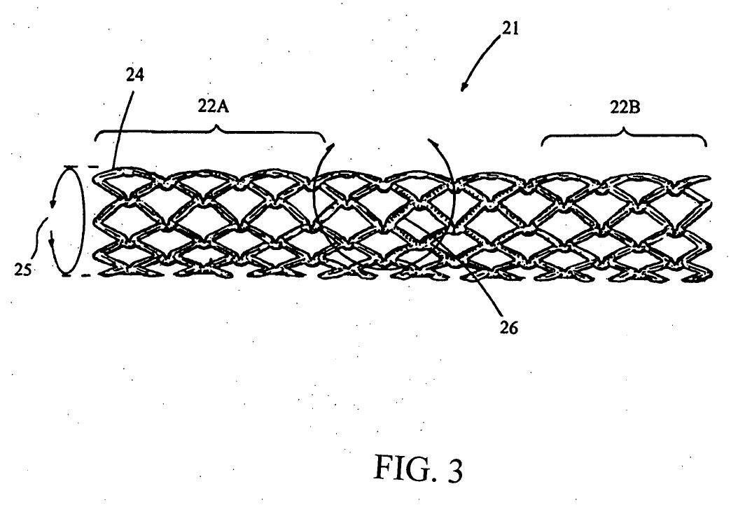

FIG. 3 shows one aspect of a biodegradable stent 21 for treating vulnerable plaques or as a retaining stent for treating an aneurysmal sac of a patient comprising at least one zone, wherein a first supporting zone 22A or 22B comprises at least a portion of continuous circumference (indicated by item 25) of the stent 21, the supporting zone being made of a first biodegradable material 24. The stent may optionally comprise a second zone at about the middle portion of the stent made of a second biodegradable material 26. In another aspect of the invention, the biodegradation rate (BR₂) of the second biodegradable material 26 of the biodegradable stent 21 is equal to or slower than the biodegradation rate (BR₁) of the first biodegradable material 24. In a particular embodiment, the first biodegradable material and/or the second biodegradable material is a shape memory polymer or a biological material with moisture memory of the present invention. In one alternate embodiment, the first zone is loaded with at least a first bioactive agent. In a further embodiment, the second zone is loaded with at least a second bioactive agent.

U.S. Pat. No. 6,160,084, No. 6,388,043, U.S. Patent Application publication no. 2003/0055198, and no. 2004/0015187, the entire contents of which are incorporated herein by reference, disclose biodegradable shape memory polymer compositions and articles manufactured therefrom. The compositions include at least one hard segment and at least one soft segment. At least one of the hard or soft segments can contain a crosslinkable group, and the segments can be linked by formation of an interpenetrating network or a semi-interpenetrating network, or by physical interactions of the segments. Objects can be formed into a given shape at a temperature above the transition temperature of the hard segment, and cooled to a temperature below the transition temperature of the soft segment. If the object is subsequently formed into a second shape, the object can return to its original shape by heating the object above the transition temperature of the soft segment and below the transition temperature of the hard segment.

Igaki and Tamai et al. in U.S. Pat. Nos. 5,733,327, 6,045,568, and 6,080,177, the entire contents of which are incorporated herein by reference, disclose luminal stents having a holding structure made of knitted yarns of biodegradable polymer fibers that subsequently disappear by being absorbed into the living tissue. Further, Igaki in U.S. Pat. Nos. 6,200,335 and No. 6,632,242, the entire contents of which are incorporated herein by reference, discloses a stent having a main mid portion and low tenacity portions formed integrally with both ends of the main mid portion. These low tenacity portions are formed so as to have the Young's modulus approximate to that of the vessel of the living body in which is inserted the stent, so that, when the stent is inserted into the vessel, it is possible to prevent stress concentrated portions from being produced in the vessel.

Further, the biological material may be selected from a group consisting of collagen, gelatin, fibrin glue, biological sealant, elastin, chitosan, N, O, carboxylmethyl chitosan, and mixture thereof, wherein the biological material is a solidifiable substrate, and wherein the biological material may be solidifiable from a phase selected from a group consisting of solution, paste, gel, suspension, colloid, and plasma. In some aspects, the biodegradable material of the biodegradable stent is made of a material selected from a group consisting of polylactic acid (PLA), polyglycolic acid (PGA), poly (D,L-lactide-co-glycolide), polycaprolactone, and co-polymers thereof. In another aspect, the biodegradable material of the biodegradable stent is made of a material selected from a group consisting of polyhydroxy acids, polyalkanoates, polyanhydrides, polyphosphazenes, polyetheresters, polyesteramides, polyesters, and polyorthoesters.

The biodegradable stent may comprise at least one bioactive agent. In one aspect, the at least one bioactive agent is selected from a group consisting of analgesics/antipyretics, antiasthamatics, antibiotics, antidepressants, antidiabetics, antifungal agents, antihypertensive agents, anti-inflammatories, antineoplastics, antianxiety agents, immunosuppressive agents, antimigraine agents, sedatives/hypnotics, antipsychotic agents, antimanic agents, antiarrhythmics, antiarthritic agents, antigout agents, anticoagulants, thrombolytic agents, antifibrinolytic agents, antiplatelet agents and antibacterial agents, antiviral agents, antimicrobials, and anti-infectives. In another aspect, the at least one bioactive agent is selected from a group consisting of actinomycin D, paclitaxel, vincristin, methotrexate, and angiopeptin, batimastat, halofuginone, sirolimus, tacrolimus, everolimus, tranilast, dexamethasone, ABT-578 (manufactured by Abbott Laboratories), and mycophenolic acid. In still another aspect, the at least one bioactive agent is selected from a group consisting of lovastatin, thromboxane A₂ synthetase inhibitors, eicosapentanoic acid, ciprostene, trapidil, angiotensin convening enzyme inhibitors, aspirin, and heparin. In a further aspect, the at least one bioactive agent is selected from a group consisting of allicin, ginseng extract, flavone, ginkgo biloba extract, glycyrrhetinic acid, and proanthocyanides. In some aspect, the at least one bioactive agent comprises ApoA-I Milano or recombinant ApoA-I Milano/phospholipid complexes. In one aspect, the at least one bioactive agent comprises biological cells or endothelial progenitor cells. In some aspects, the at least one bioactive agent comprises lipostabil. In some aspects, the at least one bioactive agent comprises a growth factor, wherein the growth factor is selected from a group consisting of vascular endothelial growth factor, transforming growth factor-beta, insulin-like growth factor, platelet derived growth factor, fibroblast growth factor, and combination thereof.

The biodegradable device can be fabricated by extrusion, molding, welding, weaving of fibers, or the like. A preferred method for making a biodegradable stent can be casting, solution molding or thermal molding, which is well known to one skilled in the art, such as exemplified in U.S. Pat. No. 6,200,335.

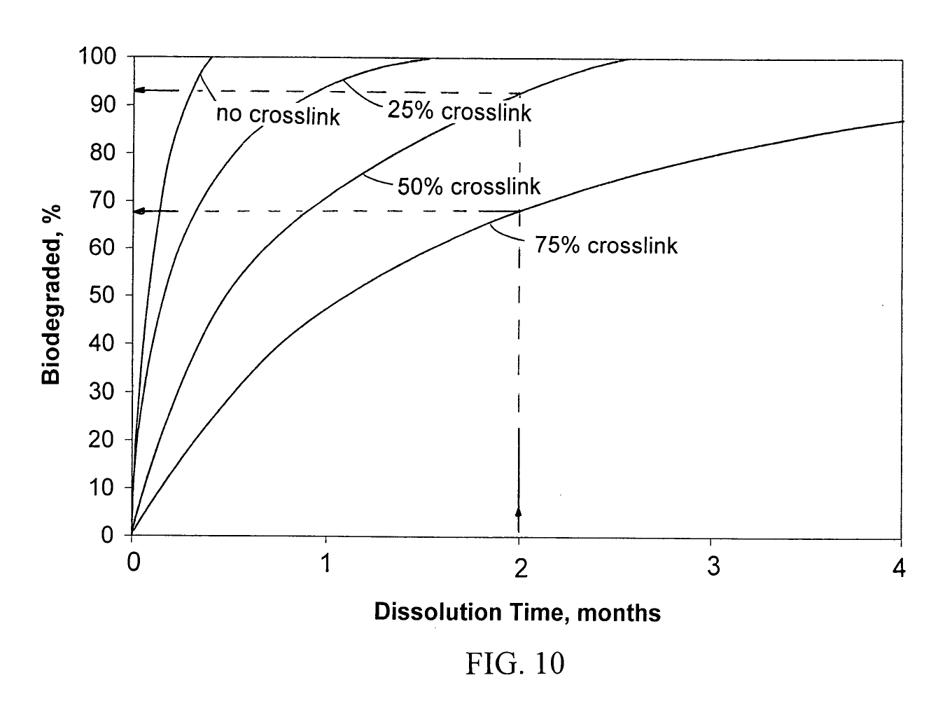

FIG. 10 shows some general trends of controlled bioresorption rates of a biodegradable device with respect to a parameter of degrees of crosslink. Some aspects of the invention relate to a flexible elongate biodegradable device for treating an aneurysm of a patient, the device being characterized with a moisture memory and a controlled biodegradation, wherein the device comprises a first configuration in a wet state sized and configured to snugly fill an aneurysm sac of the aneurysm; the device having a second configuration in a dry state configured to be loaded in a delivery catheter; and the device reversing to the first configuration after being deployed from the catheter into the sac. In FIG. 10, the biodegradation curves at various % crosslink would not cross over each other; instead, all curves follow somewhat parallel curved shape. By “controlled biodegradation”, it is meant herein that a target percent biodegradation at a specific dissolution time can be designed and configured from FIG. 10 according to a predetermined % crosslink. For example, for a biodegradable device to biodegrade at about 70% in 2 months, the device should have a % crosslink at about 75%. If we happen to use a biodegradable device with 25% crosslink, the device would biodegrade about 92% in 2 months. Hydrogel or other hydrophilic material do have biodegradation properties; however, their biodegradation is not controllable for intended purposes. The optimal time duration for an embolization device to massively biodegrade is about 2 months in situ when the embolization process establishes desired aneurysmal protection.

Suitable biodegradable polymer to be used in the present invention can be found in Handbook of Biodegradable Polymers by Domb et al. (Harwood Academic Publishers: Amsterdam, The Netherlands 1997). Some aspects of the invention provide, in combination, biodegradable and/or bioresorbable polymer as drug carrier and partially crosslinked collagen drug carrier in a drug-eluting stent of the present invention. Some aspects of the invention relate to a medical device, comprising: a biodegradable apparatus having a surface; at least one bioactive agent; and biological material loaded onto at least a portion of the surface of the apparatus, the biological material comprising the at least one bioactive agent, wherein the biological material is crosslinked with a crosslinking agent or with ultraviolet irradiation.

Suitable biodegradable polymer may comprise polylactic acid (PLA), polyglycolic acid (PGA), poly (D,L-lactide-co-glycolide), polycaprolactone, hyaluric acid, adhesive proteins, and co-polymers of these materials as well as composites and combinations thereof and combinations of other biodegradable material. Preferably the materials have been approved by the U.S. Food and Drug Administration. The differentiation of collagen from a biodegradable polymer as a drug carrier is that collagen is crosslinkable after being loaded onto a stent while the polymer is not crosslinkable any more.

One preferred aspect of the invention provides a method of treating an aneurysm sac of a patient, comprising steps of: (a) providing a flexible elongate biodegradable device with a moisture memory and a controlled biodegradation, wherein the device comprises a first configuration in a wet state sized and configured to snugly fill the aneurysm sac; (b) delivering the device to about the aneurysm sac, wherein the device comprises a second configuration in a dry state configured to be loaded in a delivery catheter during the delivery step; (c) deploying the device at the aneurysm sac, wherein the device reversely transforms to the first configuration after being deployed from the catheter; and (d) the device starting a process of biodegradation following the controlled biodegradation. In one embodiment, the method further comprises a step of placing a retaining stent at a neck of the aneurysm sac configured for preventing the device from being inadvertently dislodged from the sac, wherein the stent is biodegradable.

### EXAMPLE #7

In one aspect, the retaining stent as prepared in examples of the invention is made of a material selected from a group consisting of stainless steel, Nitinol, cobalt-chromium alloy, other cobalt containing alloy, shape memory metal, biodegradable polymer, non-biodegradable polymer, shape memory polymer, or the like. In this example, the stent is further coated with PC (phosphorylcholine). In a further embodiment, the PC coating is at least on the inner surface (that is, the blood contacting side after implanted in a blood vessel) of the stent. In another embodiment, the PC coating is at least on the outer surface (that is, the tissue contacting side after implanted in a blood vessel) of the stent. In still another embodiment, the PC coating is over the entire surface of the stent.

PC is found in the inner and outer layers of cell membrane. However, it is the predominant component present in the outer membrane layer, and because it carries both a positive and negative charge (zwitterionic), it is electrically neutral. As a result, the outer layer of the cell membrane does not promote clot formation. When PC is coated on or incorporated on a material, protein and cell adhesion is decreased, clot formation is minimized, inflammatory response is lessened, and fibrous capsule formation is minimized. Some aspects of the invention relate to a drug-eluting stent comprising an immobilized antibody (such as CD34 or the like) that attracts endothelial progenitor cells from the circulating blood stream, resulting in endothelial coverage over and between the stent struts. In a further embodiment, the antibody loading is at least on the inner surface, at least on the outer surface, or over the entire surface of the stent.

From the foregoing description, it should now be appreciated that a novel and unobvious process for making a crosslinked flexible biodegradable material as an embolization device for treating an aneurysm sac in a patient, followed by controlled bioresorption of the device in situ has been disclosed. While the invention has been described with reference to a specific embodiment, the description is illustrative of the invention and is not to be construed as limiting the invention. Various modifications and applications may occur to those who are skilled in the art, without departing from the true spirit and scope of the invention.

What is claimed is:

1. A flexible bioresorbable biological material comprising a moisture memory and a controlled bioresorption, wherein the material is crosslinked with a crosslinking agent having a degree of crosslink that is correlated to a controllable bioresorption rate configured to enable the controlled bioresorption.

2. The material according to claim 1, wherein the material with said moisture memory is in a first shape at a wet state, re-configurable to a second shape at a dry state, and reversible to said first shape after contacting moisture.

3. The material according to claim 1, wherein the biological material is selected from a group consisting of collagen, gelatin, elastin, chitosan, NOCC, chitosan-alginate complex, and combinations thereof.

4. The material according to claim 3, wherein the material is crosslinked with a crosslinking agent selected from a group consisting of genipin, its analog, derivatives, and combination thereof, aglycon geniposidic acid, epoxy compounds, and combinations thereof.

5. The material according to claim 3, wherein the material is crosslinked with a crosslinking agent selected from a group consisting of dialdehyde starch, glutaraldehyde, formaldehyde, dimethyl suberimidate, carbodiimides, succinimidyls, diisocyanates, reuterin, acyl azide, and combinations thereof.

6. The material according to claim 3, wherein the material is crosslinked with a crosslinking agent, said crosslinking agent comprises at least one ether group.

7. The material according to claim 3, wherein the material is crosslinked with a crosslinking agent, said crosslinking agent comprises ethylene glycol diglycidyl ether.

8. The material according to claim 1, further comprising at least one bioactive agent.

9. A flexible elongate biodegradable device for treating an aneurysm of a patient, the device being characterized with a moisture memory and a controlled biodegradation, wherein the device comprises a first configuration in a wet state sized and configured to snugly fill an aneurysm sac of the aneurysm; the device having a second configuration in a dry state configured to be loaded in a delivery catheter; and the device reversing to said first configuration after being deployed from the catheter into said sac.

10. The device according to claim 9, wherein the device is made of a polymer material containing at least one amino group, said material being crosslinked with a crosslinking agent having a degree of crosslink that is correlated to a controllable biodegradation rate configured to enable the controlled biodegradation.

11. The device according to claim 9, wherein the device is made of a biological material selected from a group consisting of collagen, gelatin, elastin, chitosan, NOCC, chitosan-alginate complex, and combinations thereof.

12. The device according to claim 11, further comprising at least one bioactive agent.

13. The device according to claim 11, further comprising at least one blood occluding agent.

14. The device according to claim 11, wherein the biological material is crosslinked with a crosslinking agent having a degree of crosslink, the degree of crosslink being correlated to a controllable biodegradation rate configured to enable the controlled biodegradation.

15. The device according to claim 14, wherein the crosslinking agent is selected from a group consisting of genipin, its analog, derivatives, and combination thereof, aglycon geniposidic acid, epoxy compounds, and combinations thereof.

16. The device according to claim 14, wherein the crosslinking agent is selected from a group consisting of dialdehyde starch, glutaraldehyde, formaldehyde, dimethyl suberimidate, carbodiimides, succinimidyls, diisocyanates, reuterin, acyl azide, and combinations thereof.

17. The device according to claim 14, wherein the crosslinking agent comprises at least one ether group.

18. The device according to claim 14, wherein the crosslinking agent is ethylene glycol diglycidyl ether.

19. A method of treating an aneurysm sac of a patient, comprising steps of:
providing a flexible elongate biodegradable device with a moisture memory and a controlled biodegradation, wherein the device comprises a first configuration in a wet state sized and configured to snugly fill the aneurysm sac;
delivering the device to about the aneurysm sac, wherein the device comprises a second configuration in a dry state configured to be loaded in a delivery catheter during the delivering step;
deploying the device at the aneurysm sac, wherein the device reversely transforms to said first configuration after being deployed from the catheter; and
the device starting a process of biodegradation following said controlled biodegradation of the device.

20. The method according to claim 19, further comprising a step of placing a retaining stent at a neck of said aneurysm sac configured for preventing the device from being inadvertently dislodged from said sac, wherein the stent is biodegradable.
